(19) United States
(12) Patent Application Publication
(10) Pub. No.: US 2008/0006530 A1
(43) Pub. Date: Jan. 10, 2008

(54) Capillary Flow Control in a Flow Channel

(76) Inventors: Handani Winarta, Nashua, NH (US); Chung Chang Young, Weston, MA (US); Andy Vo, Somerville, MA (US); Yu-Tao Chuang, Jhonghe City (TW); Yu-Feng Chang, Yonghe City (TW)

Correspondence Address: MESMER & DELEAULT, PLLC, 41 BROOK STREET, MANCHESTER, NH, 03104, US

(21) Appl. No.: 11/425,001

(22) Filed: Jun. 19, 2006

(51) Int. Cl. G01N 33/487

(52) U.S. Cl. 204/403.01

(57) ABSTRACT

A sensor strip with capillary flow control has a base layer having a plurality of conductive paths delineated thereon, an electrode forming layer disposed on the base layer, the electrode forming layer having a first opening forming a working electrode, a second opening forming a reference electrode, and a flow-control mechanism, a spacer layer disposed on the electrode forming layer, and a cover layer with a vent opening disposed on the spacer layer and forming a substantially flat sample channel with walls formed by the electrode forming layer, the spacer layer and the cover layer, the substantially flat sample chamber having a sample inlet adjacent a proximal end and the vent opening adjacent a distal end where the substantially flat sample chamber contains the working electrode, the reference electrode and the flow-control mechanism.

### BACKGROUND OF THE INVENTION

1. Field of the Invention

The present invention relates generally to controlling capillary flow in a flow channel. Particularly, the present invention relates to controlling capillary flow in a flow channel in a sensor strip.

2. Description of the Prior Art

Controlling capillary flow in a flow channel is important in microfluidic and microanalytical systems. This is of particular importance for sensor strips used in determining blood analytes such as, for example, blood glucose. It is well known that the concentration of blood glucose is extremely important for maintaining homeostasis. Products that measure fluctuations in a person's blood sugar, i.e. glucose level, have become everyday necessities for many of the nation's millions of diabetics. Because this disorder can cause dangerous anomalies in blood chemistry and is believed to be a contributor to vision loss and kidney failure, most diabetics need to test themselves periodically and adjust their glucose level accordingly, usually with insulin injections. If the concentration of blood glucose is below the normal range, patients can suffer from unconsciousness and lowered blood pressure which may even result in death. If the blood glucose concentration is higher than the normal range, the excess blood glucose can result in synthesis of fatty acids and cholesterol, and in diabetics, coma. Thus, the measurement of blood glucose levels has become a daily necessity for diabetic individuals who control their level of blood glucose by insulin therapy.

Patients who are insulin dependent are instructed by doctors to check their blood-sugar levels as often as four times or more a day. To accommodate a normal life style to the need of frequent monitoring of glucose levels, home blood glucose testing was made available with the development of reagent strips for whole blood testing.

One type of blood glucose biosensor is an enzyme electrode combined with a mediator compound which shuttles electrons between the enzyme and the electrode resulting in a measurable current signal when glucose is present. The most commonly used mediators are potassium ferricyanide, ferrocene and its derivatives, as well as other metal-complexes. Many sensors based on this second type of electrode have been disclosed.

Blood glucose testing systems have undergone various improvements that have reduced the time to make a blood glucose measurement from 1-2 minutes down to 5 seconds. Some of these systems are known as the Accu-Chek® Aviva system by Roche Diagnostics, the One-Touch® system by LifeScan, the Glucometer® DEX system by Bayer, the True Track® system by Home Diagnostics, the Freestyle® system by Abbott, and BD Logic® Blood Glucose Monitor by BD Diagnostics. Introduction of a liquid sample to these sensor strips can be achieved in several ways. A simple approach is to place a sample of liquid directly onto the reaction site. A second approach is to define a cavity having dimensions small enough to allow the liquid sample to be taken up by capillary attraction. An alternative to the use of capillary attraction is to place a mesh in the sample path to aid in transporting the sample by wicking action to fill the reaction site.

However, these blood glucose testing systems are less accurate than they could be. It is widely acknowledged that the cause of the inaccuracy, though small, arises because the capillary flow of the sample is still moving while the test measurement is being taken.

One attempt to provide a mechanism to stop the movement of the liquid sample is disclosed in U.S. Pat. No. 6,939,450.

U.S. Pat. No. 6,939,450 (2005, Karinka et al.) discloses device having a flow channel where at least one flow-terminating interface is used to control the flow of liquid in the flow channel. The flow terminating interface prevents the flow of the liquid beyond the interface. In one aspect, the invention provides a sensor, such as, for example, a biosensor in the form of a strip, the sensor being suitable for electrochemical or optical measurement. The sensor comprises a base layer and a cover layer. The base layer is separated from the cover layer by a spacer layer. The base layer, cover layer and spacer layer define a flow channel into which a liquid sample is drawn therein and flows therethrough by means of capillary attraction. The flow-terminating interface is either a hydrophobic barrier in the flow channel positioned after the hydrophilic portion of the flow channel or the flow channel is closed at the distal end and has no openings in the sidewalls but includes at least one, but preferably a plurality of openings in the cover layer that serve to vent air from the flow channel and act as the flow-terminating interface.

A disadvantage of the former embodiment is the need to make the portion of the flow channel containing the test measuring region hydrophilic while making the portion of the flow channel after the test measuring region hydrophobic. This requires greater care and detail in making the sensor so that it has two distinct regions, one after the other, within the flow channel. A disadvantage of the latter embodiment is that in some strips, the sample may continue to creep beyond the flow terminating interface because of the hydrophilic character of the flow channel. In order to stop the creep the end beyond the holes in the cover is closed.

Therefore, what is needed is a flow terminating mechanism that controls the flow of liquid in a capillary flow channel without maintaining a careful separation within the flow channel between a hydrophilic test region within the flow channel from a hydrophobic region beyond the test region in the flow channel. What is also needed is a flow terminating mechanism that can eliminate sample creep beyond the vent openings without resorting to a closed end channel.

### SUMMARY OF THE INVENTION

It is an object of the present invention to provide a flow terminating mechanism within a flow channel without the need to maintain a careful separation within the flow channel between a hydrophilic test region from a hydrophobic region beyond the test region in the flow channel. It is another object of the present invention to provide a flow terminating mechanism that can eliminate sample creep beyond the vent openings without resorting to a closed end channel beyond the vent openings. It is a further object of the present invention to provide a test strip having a substantially flat sample chamber with a flow terminating mechanism within the sample chamber that is simple to create and does not require a careful and meticulous manufacturing process.

The present invention achieves these and other objectives by providing a flow channel with a flow terminating mechanism that does not require a separate and distinct hydrophilic region within the flow channel followed by a separate and distinct hydrophobic region after the hydrophilic region. Specifically, the flow channel has either one entire wall of the flow channel that is hydrophobic or has a recess in one wall after the sample containing region, or both. A sensor strip incorporating the flow terminating mechanism of the present invention may be based on amperometric, coulometric, potentiometric, voltammetric, and other electrochemical techniques as well as optical techniques for determining the concentration of an analyte in a sample. Specifically, the sensor strip includes a laminated body, a fluid sampling end with a sample inlet, a vent opening, and a sample chamber between the sample inlet and the vent opening.

In a sensor strip embodiment based on electrochemical techniques, the sensor strip includes an electrical contact end. The laminated body includes a base layer with a plurality of electrically conductive paths, an electrode forming layer with a plurality of electrode openings and a flow terminating mechanism, a spacer layer, and a cover layer with a vent opening. The electrically conductive paths may be made from any non-corroding metal. Carbon deposits such as for example carbon paste or carbon ink may also be used as the conductive paths, all as is well known by those of ordinary skill in the art.

The plurality of electrode openings of the electrode forming layer form electrode wells in the sample chamber of the sensor strip when the electrode forming layer is assembled to the base layer. The electrode wells hold chemical reagents forming one or more working, reference and/or other interference correcting electrodes such as, for example, glucose measuring strips. The flow terminating mechanism of electrode forming layer is a hydrophobic coating, a flow terminating recess, or both.

Spacer layer has an extended slot at the fluid sampling end that forms the side walls of the sample chamber. The electrode wells and the flow terminating mechanism lie within the extended slot or cutout of the spacer layer.

The cover layer completes the formation of the sample chamber, which is a substantially flat sample chamber. At least a portion of the vent opening communicates with the sample chamber to allow air in the chamber to escape when a fluid sample enters the sample chamber by capillary action and displaces the air.

In the embodiment with the flow terminating recess in the electrode forming layer, the presence of the flow terminating recess provides sensor strips capable of more accurate measurements. It is important that the flow terminating recess always be the furthest downstream from the sample inlet after the electrode wells.

In the embodiment with the hydrophobic coating, the entire electrode forming layer exposed in the sample chamber has the hydrophobic coating, or is made from a hydrophobic material. In unmodified sensor strips, i.e. sensor strips without a flow terminating mechanism, the liquid sample entering and filing the sample chamber by capillary action typically flows up to the edge of the vent opening in the cover layer. Some of these unmodified strips experience sample creep, i.e. the liquid sample continues to creep past the edge of the vent opening.

If sample creep occurs during the time that a sample measurement is being taken, error in the measurement is introduced. The hydrophobic coating acts to reduce the momentum of the liquid sample as the sample chamber is being filled by capillary action. The reduced momentum of the liquid sample allows the edge of the vent opening to prevent sample creep by stopping the sample.

### BRIEF DESCRIPTION OF THE DRAWINGS

FIG. 1 is a perspective view of a sensor strip of the present invention showing the various layers of the laminated body.

FIG. 2 is a perspective view of one embodiment of the present invention showing the various layers of the laminated body and the flow terminating mechanism.

FIG. 3 is a perspective view of another embodiment of the present invention showing the various layers of the laminated body and the flow terminating mechanism.

### DETAILED DESCRIPTION OF THE PREFERRED EMBODIMENT

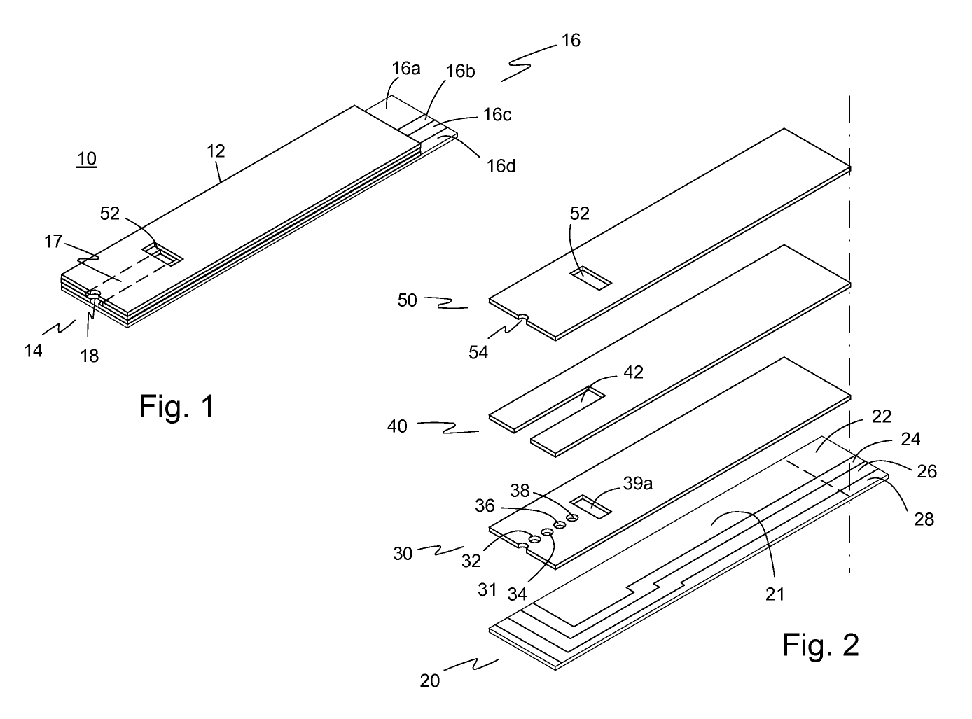

The preferred embodiments of the present invention are illustrated in FIGS. 1-3. FIG. 1 shows one embodiment of a sensor strip 10 of the present invention. In this embodiment, sensor strip 10 has a laminated body 12, a fluid sampling end 14, an electrical contact end 16, and a vent opening 52. Fluid sampling end 14 includes a sample chamber 17 between a sample inlet 18 and vent opening 52. Electrical contact end 16 has four discrete conductive contacts 16a, 16b, 16c and 16d. Sample chamber 17 is a substantially flat sample chamber with a proximal end adjacent the sample inlet 18 and a distal end adjacent the vent opening 52.

Turning now to FIG. 2, laminated body 12 is composed of a base layer 20, a electrode forming layer 30, a spacer layer 40, and a cover layer 50. All layers of laminated body 12 are made of a dielectric material, preferably plastic. Examples of a preferred dielectric material are polyvinyl chloride, polycarbonate, polysulfone, nylon, polyurethane, cellulose nitrate, cellulose propionate, cellulose acetate, cellulose acetate butyrate, polyester, polyimide, polypropylene, polyethylene and polystyrene.

Base layer 20 has a conductive layer 21 on which are delineated four conductive paths 22, 24, 26, and 28. It should be understood that the conductive paths in any of the embodiments disclosed herein may be made from any non-corroding metal. Carbon deposits such as for example carbon paste or carbon ink may also be used as the conduit paths, all as is well known by those of ordinary skill in the art.

The conductive paths 22, 24, 26, and 28 may be formed by scribing or scoring conductive layer 21, or by silk-screening conductive paths 22, 24, 26, and 28 onto base layer 20. Scribing or scoring of conductive layer 21 may be done by mechanically scribing the conductive layer 21 sufficiently to create the independent conductive paths 22, 24, 26, and 28. The preferred scribing or scoring method of the present invention is done by using a carbon dioxide laser, a YAG laser or an eximer laser. Conductive layer 21 may be made of any electrically conductive material such as, for example, gold, tin oxide/gold, palladium, other noble metals or their oxides, or carbon film compositions. The preferred electrically conductive material is gold or tin oxide/gold. A usable material for base layer 20 is a tin oxide/gold polyester film (Cat. No. FM-1) or a gold polyester film (Cat. No. FM-2) sold by Courtaulds Performance Films, Canoga Park, Calif.

Electrode forming layer 30 has two or more electrode forming openings. In this preferred embodiment, electrode forming layer 30 has four electrode forming openings 32, 34, 36, and 38. Electrode forming opening 32 exposes a portion of conductive path 22, electrode forming opening 34 exposes a portion of conductive path 24, electrode forming opening 36 exposes a portion of conductive path 26, and electrode forming opening 38 exposes a portion of conductive path 28. Each of the openings form electrode forming wells when electrode forming layer is disposed onto base layer 20. Electrode forming layer 30 also includes a flow terminating mechanism 39. In this embodiment, flow terminating mechanism 39 is a flow terminating opening 39a that creates a recess/opening in one wall of sample chamber 17 (preferably in the wall opposite vent opening 52) when assembled into sensor strip 10. Flow terminating opening 39a is a critical aspect of the present invention that causes sensor strip 10 to provide more accurate measurements. Flow terminating opening 39a, when incorporated into sensor strip 10, is always the opening/recess on electrode forming layer 30 that is the furthest downstream from sample inlet 18. Electrode forming layer 30 also includes an optional notch 31 at fluid sampling end 14 to facilitate loading of the fluid sample into sample chamber 17. The preferred shape is a half circle, which is located approximately in the middle of the channel entrance. The preferred size is 0.028 in. (0.71 mm) in diameter.

Electrode forming layer 30 is made of a plastic material, preferably a medical grade, one-sided, adhesive tape available from Adhesive Research, Inc., of Glen Rock, Pa. Acceptable thicknesses of the tape for use in the present invention are in the range of about 0.001 in. (0.025 mm) to about 0.005 in. (0.13 mm). One such tape, Arcare® 7815 (about 0.0025 in. (0.063 mm)), is preferred due to its ease of handling and good performance. It should be understood that the use of a tape is not required. Electrode forming layer 30 may be made from a plastic sheet and may be coated with a pressure sensitive adhesive, a photopolymer, ultrasonically-bonded to base layer 20, or silk-screened onto the base layer 20 to achieve the same results as using the polyester tape mentioned.

The four electrode forming openings 32, 34, 36, and 38 define four electrode areas that can be any combination of working electrodes, reference electrodes, and/or other interference compensating electrodes. Each electrode area holds chemical reagents specific for the type of electrode desired. The chemical reagents for the working electrode areas are typically a mixture of enzymes and redox mediators with optional polymers, surfactants, and buffers. Examples of usable reagents are disclosed in U.S. Pat. Nos. 6,258,229; 6,287,451; 6,837,976; 6,942,770, which are incorporated herein by reference. A reference reagent matrix may be loaded in at least one electrode area forming a reference electrode.

Typically, the reference electrode area must be loaded with a redox reagent or mediator to make the reference electrode function when using the preferred conductive coating material. The reference reagent matrix preferably contains either oxidized or a mixture of an oxidized and reduced form of redox mediators, at least one binder, a surfactant and an optional antioxidant (if a reduced form of redox mediator is used) and a bulking agent. In the alternative, the reference electrode could be also loaded with a Ag/AgCl layer (e.g. by applying Ag/AgCl ink or by sputter-coating a Ag or Ag/AgCl layer) or other reference electrode materials that do not require a redox mediator to function properly.

The size of the electrode forming openings is preferred to be made as small as possible in order to make the sample chamber of the sensor as short as possible while still being capable of holding sufficient chemical reagent to function properly. The preferred shape of the electrode forming openings is round and has a preferred diameter of about 0.03 in. (0.76 mm). The four electrode forming openings 32, 34, 36, and 38 illustrated in FIG. 2 are aligned with each other and are spaced about 0.025 in. (0.625 mm) from each other. The circular electrode forming openings are for illustrative purposes only and it should be understood that the shape of the electrode forming openings is not critical.

The positional arrangement of the working electrode and the reference electrode in the channel is also not critical for obtaining usable results from the sensor. The position of the flow terminating opening/recess 39a, however, is critical. As disclosed above, it is important that flow terminating opening/recess 39a is the last (i.e. downstream) opening/recess in sample chamber 17 and that it is formed within the wall and across the entire width of the wall in sample chamber 17 in which it is located.

The working electrode and the reference electrode are each in electrical contact with separate conductive paths. The separate conductive paths terminate and are exposed for making an electrical connection to a reading device at electrical contact end 16 of laminated body 12.

Spacer layer 40 has a U-shaped cutout or slot 42 located at the fluid sampling end 14. The length of cutout 42 is such that when spacer layer 40 is laminated to electrode forming layer 30, the electrode areas and the flow terminating opening/recess 39a are within the space defined by cutout 42. Spacer layer 40 is made of a plastic material, preferably a medical grade, double-sided, pressure sensitive adhesive tape available from Adhesive Research, Inc., of Glen Rock, Pa. Acceptable thicknesses of the tape for use in the present invention are in the range of about 0.001 in. (0.025 mm) to about 0.010 in. (0.25 mm). One such tape is Arcare® 7840 (about 0.0035 in. (0.089 mm)). U-shaped cutout 42 can be made with a laser or by die-cutting. The preferred method is to die-cut the cutout. The preferred size of the U-shaped cutout is about 0.05 in. wide (1.27 mm) and about 0.0035 in. thick (0.089 mm). The length is dependent on the number of working, reference and other electrodes incorporated into sensor strip 10.

Cover layer 50, which is laminated to spacer layer 40, has vent opening 52 spaced from the fluid sampling end 14 of sensor strip 10 to insure that fluid sample in the sample chamber 17 will completely cover all the electrode areas. Vent opening 52 is positioned in cover layer 50 so that it will align somewhat with U-shaped cutout 42. Preferably, vent opening 52 will expose a portion of and partially overlay the base of the U-shaped cutout 42. The preferable shape of vent hole 52 is a rectangle with dimensions of about 0.08 in. (2 mm) by about 0.035 in. (0.9 mm). Preferably, the top layer also has an optional notch 54 at fluid sampling end 14 to facilitate loading of the fluid sample into sample chamber 17. The preferred shape is a half circle, which is located approximately in the middle of the channel entrance. The preferred size is 0.028 in. (0.71 mm) in diameter. The preferred material for cover layer 50 is a polyester film. In order to facilitate the capillary action, it is desirable for the polyester film to have a highly hydrophilic surface that faces the capillary channel. Transparency films (Cat. No. PP2200 or PP2500) from 3M are the preferred material used as the cover layer in the present invention.

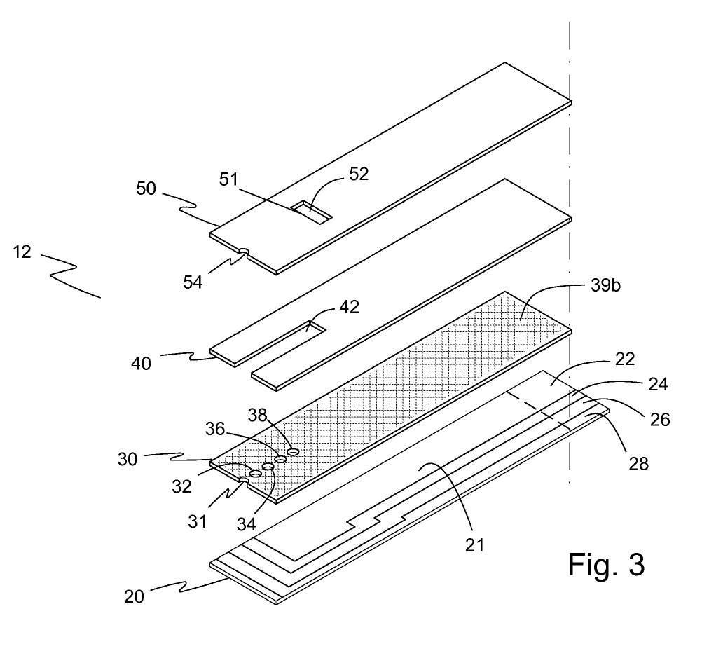

FIG. 3 illustrates an expanded view of yet another embodiment of the present invention. This embodiment also has a similar structure to the sensor strip 10 shown in FIG. 1. The difference in this embodiment is in the manner in which sample flow control is accomplished.

Laminated body 12 has a base layer 20, a electrode forming layer 30, a spacer layer 40 with a U-shaped cutout 42, and a cover layer 50 with an optional inlet notch 54. Base layer 20 has a conductive layer 21 on which is delineated a plurality of conductive paths 22, 24, 26, and 28. Electrode forming layer 30 has two or more electrode forming openings. In this embodiment, like the previous embodiment, electrode forming layer 30 has four electrode forming openings 32, 34, 36, and 38, and an optional notch 31.

Unlike the previous embodiment, this embodiment does not have a flow terminating opening/recess. Flow control is accomplished in this embodiment by a hydrophobic layer 39b on electrode forming layer 30 or by making electrode forming layer 30 using a hydrophobic material. The important aspect of the present invention is that the portion of electrode forming layer 30 lying within sample chamber 17 is made hydrophobic over its entire length. Maintaining this portion of sample chamber 17 hydrophobic reduces the momentum of the sample fluid as it enters sample chamber 17 by capillary action. The reduced momentum caused by making the electrode forming layer hydrophobic allows edge 51 of vent opening 52 to prevent sample creep by stopping the sample fluid completely.

In sensor strips having a hydrophilic or somewhat less hydrophobic sample chamber 17, it was found that the sample fluid would in some instances continue to creep along the sample chamber wall opposite vent opening 52 past the first edge 51 of vent opening 52.

Sample movement arising from this creeping of the sample fluid that occurs during the measurement reading time of sensor strip 10 introduces error in the measurement.

Making of the Sensor Strip

Assembly of the various embodiments of the present invention is relatively straightforward. Generally, the base layer and electrode forming layer are laminated to each other followed by dispensing the appropriate reagent mixture(s) into each of the electrode forming openings. After drying the reagent mixture, the spacer layer is laminated onto the electrode forming layer and the cover layer is then laminated onto the spacer layer.

More particularly, a piece of a gold polyester film is cut to shape as illustrated in FIG. 2, forming base layer 20 of sensor 10. A laser (previously disclosed) is used to score the gold polyester film. As illustrated in FIG. 2, the film is scored by the laser such that two or more electrodes at sample fluid end 14 and an equivalent number of contact points are formed at electrical contact end 16. The scoring line is very thin but sufficient to create separate electrically conductive paths. A scoring line may optionally be made, but is not necessary, along the outer edge of base layer 20 to avoid potential static problems which could cause a noisy signal from the sensor strip 10.

A piece of one-sided adhesive tape is then cut to size and shape, forming electrode forming layer 30 so that it will cover a major portion of conductive layer 21 of base layer 20 except for exposing a small electrical contact area illustrated in FIG. 1.

The flow terminating mechanism 39 and the electrode forming openings are incorporated into electrode forming layer 30. In the embodiment where flow terminating mechanism 39 is a flow terminating opening/recess 39a, flow terminating opening/recess 39a and the two or more circular openings such as those illustrated in FIG. 2 with reference numbers 32, 34, 36, and 38 are punched by laser, or by mechanical means such as a die-punch assembly, creating electrode openings 32, 34, 36, and 38, and flow terminating opening 39a in electrode forming layer 30. The shape of the openings in electrode forming layer 30 is for illustrative purposes only. It should be understood that the shape of the openings is not critical, provided that the size of the openings is big enough to hold sufficient chemical reagents for the electrodes to function properly but small enough to allow for a reasonably small sample chamber. In the embodiment where flow terminating mechanism 39 is a hydrophobic coating 39b, hydrophobic coating 39b is preferably applied to the top surface of electrode forming layer 30 before the electrode openings are formed. Various types of coatings may be used such as, for example, photoresist ink material that can be screen printed or sprayed onto the surface of electrode forming layer 30. An example of a preferred hydrophobic coating is a material sold as catalog no. SGS-925 and available from SGS in Taiwan.

Electrode forming layer 30 is then attached to base layer 20 in such a way as to define the two or more electrode wells. Approximately 0.05 to 0.09 μL of the appropriate reagent mixture (or mixtures) is dispensed into respectively appropriate electrode areas. After the addition of the reagents, the reagents are dried. Drying of the reagents can occur within a temperature range of about room temperature to about 80° C. The length of time required to dry the reagents is dependent on the temperature at which the drying process is performed.

After drying, a piece of double-sided tape available from Adhesive Research is fashioned into spacer layer 40 containing U-shaped channel 42. Spacer layer 40 is then layered onto electrode forming layer 30. As mentioned earlier, spacer layer 40 serves as a spacer and defines the size of the sample chamber 17.

A piece of a transparency film (Cat. No. PP2200 or PP2500 available from 3M) is fashioned into top layer/cover layer 50. A rectangular vent opening 52 is made using the laser previously mentioned or by means of a die-punch. Vent opening 52 is located approximately 0.180 in. (4.57 mm) from sample inlet 18. Cover layer 50 is aligned and layered onto spacer layer 40 to complete the assembly of sensor 10, as illustrated in FIG. 1.

Those skilled in the art, however, will recognize that a sensor strip incorporating the flow terminating mechanism of the present invention may be utilized in sensor strips based on amperometric, coulometric, potentiometric, voltammetric, and other electrochemical techniques as well as optical techniques for determining the concentration of an analyte in a sample.

The following examples illustrate the unique features of the present invention. All three examples used the same aqueous control samples. Control 1 is a low level control containing a glucose concentration of 60±15 mg/dL. Control 2 is a normal level control containing a glucose concentration of 120±25 mg/dL. Control 3 is a high level control containing a glucose concentration of 300±35 mg/dL. The meter used to make the measurements in the examples is a prototype glucose meter based on amperometry and made by Nova Biomedical Corporation, Waltham, Mass.

### EXAMPLE 1

### Demonstration of Response at Different Glucose Levels for an Unmodified Sensor Strip

Control samples with different glucose concentrations were tested with the glucose measuring strips manufactured by Nova Biomedical Corporation and having the laminated structure disclosed in the preferred embodiment and connected to the prototype meter. These strips did not have any flow terminating mechanism within the sample chamber. Table 1 shows the data obtained using the single use glucose measuring sensor strips at each of three control levels of glucose. A total of 20 measurements were made for each control sample.

TABLE 1

| | Control 1 | Control 2 | Control 3 |
|---|---|---|---|
| | 63 | 183 | 366 |
| | 61 | 171 | 347 |
| | 65 | 178 | 345 |
| | 64 | 162 | 353 |
| | 55 | 173 | 356 |
| | 66 | 175 | 349 |
| | 40 | 180 | 353 |
| | 66 | 163 | 356 |
| | 67 | 156 | 331 |
| | 67 | 112 | 329 |
| | 51 | 178 | 356 |
| | 65 | 178 | 352 |
| | 61 | 176 | 335 |
| | 70 | 176 | 322 |
| | 44 | 180 | 222 |
| | 64 | 174 | 348 |
| | 62 | 171 | 241 |
| | 40 | 174 | 332 |
| | 63 | 159 | 353 |
| | 64 | 175 | 361 |
| Mean | 59.9 | 169.7 | 335.4 |
| C.V. | 15.1% | 9.1% | 11.2% |

As shown in Table 1, the coefficients of variation for the three control groups were 15.1, 9.1 and 11.2, respectively.

### EXAMPLE 2

### Demonstration of Response at Different Glucose Levels for Modified Sensor Strip with Hydrophobic Coating as the Flow Terminating Mechanism

Control samples with different glucose concentrations were tested with modified glucose strips manufactured by Nova Biomedical Corporation. The modified strips had the laminated structure disclosed in the preferred embodiment and further incorporated a hydrophobic coating 39b as the flow terminating mechanism 39 illustrated in FIG. 3. These modified sensor strips were tested using the prototype meter disclosed above. Table 2 shows the data obtained using the single use glucose measuring sensor strips at each of three control levels of glucose. A total of 20 measurements were made for each control sample.

TABLE 2

| | Control 1 | Control 2 | Control 3 |
|---|---|---|---|
| | 70 | 183 | 365 |
| | 67 | 179 | 357 |
| | 66 | 180 | 346 |
| | 67 | 185 | 336 |
| | 72 | 187 | 361 |
| | 66 | 182 | 363 |
| | 63 | 186 | 362 |
| | 67 | 167 | 357 |
| | 68 | 174 | 347 |
| | 66 | 179 | 339 |
| | 71 | 183 | 350 |
| | 68 | 183 | 336 |
| | 68 | 188 | 350 |
| | 69 | 182 | 328 |
| | 68 | 171 | 344 |
| | 73 | 171 | 363 |
| | 67 | 182 | 345 |
| | 65 | 190 | 352 |
| | 69 | 181 | 337 |
| | 68 | 174 | 339 |
| Mean | 67.9 | 180.4 | 348.9 |
| C.V. | 3.5% | 3.4% | 3.1% |

As shown in Table 2, the coefficients of variation for the three control groups were 3.5, 3.4 and 3.1, respectively. When compared to the unmodified sensor strips, it was found that the sensor strips having the hydrophobic coating as the flow terminating mechanism had less error in the measurements (i.e. better C.V.) and were more consistent than the unmodified strips.

### EXAMPLE 3

### Demonstration of Response at Different Glucose Levels for Modified Sensor Strip with Flow Terminating Recess

Control samples with different glucose concentrations were tested with modified glucose strips manufactured by Nova Biomedical Corporation. The modified strips had the laminated structure disclosed in the preferred embodiment and incorporated a flow terminating recess 39a in the electrode forming layer 20 as the flow terminating mechanism 39 illustrated in FIG. 2. These modified sensor strips were tested using the previously described prototype meter. Table 2 shows the data obtained using the single use glucose measuring sensor strips at each of three control levels of glucose. A total of 20 measurements were made for each control sample.

TABLE 3

| | Control 1 | Control 2 | Control 3 |
|---|---|---|---|
| | 69 | 176 | 347 |
| | 68 | 182 | 344 |
| | 74 | 188 | 359 |
| | 71 | 181 | 355 |
| | 69 | 180 | 347 |
| | 68 | 178 | 342 |
| | 71 | 176 | 333 |
| | 68 | 179 | 345 |
| | 69 | 176 | 342 |
| | 72 | 182 | 346 |
| | 68 | 177 | 340 |
| | 68 | 185 | 347 |
| | 71 | 187 | 359 |
| | 67 | 184 | 351 |
| | 69 | 175 | 346 |
| | 70 | 176 | 338 |
| | 68 | 182 | 350 |
| | 70 | 179 | 345 |
| | 70 | 176 | 345 |
| | 67 | 178 | 349 |
| Mean | 69.4 | 179.9 | 346.5 |
| C.V. | 2.6% | 2.2% | 1.8% |

As shown in Table 3, the coefficients of variation for the three control groups were 2.6, 2.2 and 1.8, respectively. As illustrated in Table 3, the coefficients of variation are improved compared to the coefficients of variation resulting from the use of a hydrophobic coating 39b as the flow terminating mechanism 39 and is significantly improved compared to the coefficients of variation for the sensor strips having no flow terminating mechanism. The flow terminating mechanism 39 used in Example 3 provided measurement values having the least amount of error in the measurements (i.e. best C.V.) and were the most consistent.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. A sensor strip with capillary flow control, said sensor strip comprising:
a base layer having a plurality of conductive paths delineated thereon;
an electrode forming layer disposed on said base layer, said electrode forming layer having a first opening forming a working electrode, a second opening forming a reference electrode, and a flow-control mechanism;
a spacer layer disposed on said electrode forming layer; and
a cover layer with a vent opening disposed on said spacer layer and forming a substantially flat sample channel with walls formed by said electrode forming layer, said spacer layer and said cover layer, said substantially flat sample chamber having a sample inlet adjacent a proximal end and said vent opening adjacent a distal end wherein said substantially flat sample chamber contains said working electrode, said reference electrode and said flow-control mechanism.

2. The sensor strip of claim 1 wherein said flow control mechanism is a coating on said electrode forming layer over the length of the sample chamber that slows the momentum of a liquid sample sufficiently to stop the migration of said liquid sample at the edge of said vent opening.

3. The sensor strip of claim 2 wherein said coating is a hydrophobic coating.

4. The sensor strip of claim 1 wherein said flow control mechanism is a recess formed in said electrode forming layer.

5. The sensor strip of claim 4 wherein said recess spans across the width of said sample chamber.

6. The sensor strip of claim 4 wherein said recess is a mirror-image of said vent opening.

7. The sensor strip of claim 1 wherein said electrode forming layer has a notch at a distal end adjacent said sample inlet.

8. A sensor strip with capillary flow control, said sensor strip comprising:
a substantially flat sample chamber having a sample inlet, a vent opening, a top wall, a bottom wall, and side walls wherein the surface area of said top wall and said bottom wall is substantially greater than the surface area of said side walls; and
a flow terminating mechanism within said substantially flat sample chamber, said flow terminating mechanism being integral with said bottom wall.

9. The sensor strip of claim 8 wherein said flow terminating mechanism is a hydrophobic coating over the entire surface of said bottom wall.

10. The sensor strip of claim 8 wherein said flow terminating mechanism is a recess in and across the width of said bottom wall, said recess positioned downstream from said sample inlet.

11. A capillary flow channel with a flow terminating mechanism, said capillary flow channel comprising:
a substantially flat sample chamber having a bottom wall, a top wall, and side walls with a sample inlet at a proximal end of said substantially flat sample chamber and a vent opening in said top wall at a distal end of said substantially flat sample chamber; and
a flow terminating mechanism integrally formed with said bottom wall, said flow terminating mechanism being within said capillary flow channel.

12. The capillary flow channel of claim 11 wherein said flow terminating mechanism is a hydrophobic coating over the entire surface of said bottom wall.

13. The capillary flow channel of claim 11 wherein said flow terminating mechanism is a recess in and across the width of said bottom wall, said recess positioned downstream from said sample inlet.
